(19) United States
(12) Patent Application Publication
(10) Pub. No.: US 2025/0139774 A1
(43) Pub. Date: May 1, 2025

(54) METHODS AND SYSTEMS FOR MACHINE LEARNING-BASED PREDICTION OF GENE ALTERATIONS FROM PATHOLOGY IMAGES

(71) Applicant: Foundation Medicine, Inc., Boston, MA (US)

(72) Inventors: James PAO, Boston, MA (US); Mikayla BIGGS, Boston, MA (US)

(73) Assignee: Foundation Medicine, Inc., Boston, MA (US)

(21) Appl. No.: 18/924,829

(22) Filed: Oct. 23, 2024

Related U.S. Application Data
(60) Provisional application No. 63/546,060, filed on Oct. 27, 2023.

(51) Int. Cl. G06T 7/00; G06T 7/11; G06V 10/764; G06V 20/69; G16H 30/20; G16H 30/40; G16H 50/20

(52) U.S. Cl. CPC G06T 7/0012; G06T 7/11; G06V 10/764; G06V 20/698; G16H 30/20; G16H 30/40; G16H 50/20; G06T 2207/30024; G06T 2207/30096

(57) ABSTRACT

Methods for predicting gene alteration states in tissue samples based on analysis of pathology images are described. The methods may comprise, e.g., receiving a plurality of image patches derived from at least one slide image, where each image patch is associated with a tissue phenotype classification label; identifying a set of tumor region image patches based on the tissue phenotype classification label for each image patch of the plurality; identifying buffer region image patches from the plurality of image patches based on the identified set of tumor region patches and a proximity relationship; inputting the tumor region and buffer region image patches into a gene alteration state classification model configured to predict a gene alteration state based on image features identified in the tumor region and buffer region image patches; and outputting a prediction of at least one gene alteration state for the tissue sample.

### CROSS-REFERENCE TO RELATED APPLICATIONS

This application claims the priority benefit of U.S. Provisional Patent Application Ser. No. 63/546,060, filed Oct. 27, 2023, the content of which is incorporated herein by reference in its entirety.

### FIELD OF THE INVENTION

The present disclosure relates generally to methods and systems for predicting gene alterations in a sample, e.g., a tissue sample, using a machine learning-based analysis of histological (e.g., pathological) whole slide images.

### BACKGROUND

Identification of the presence of certain gene alterations in tumors (e.g., using a molecular assay or sequencing technique) can produce a wide range of clinically impactful information (e.g., diagnostic, prognostic, and/or predictive information) that may be used to provide patients with increasingly personalized care to improve healthcare outcomes. For example, a common usage of molecular assays for lung adenocarcinoma patients is to determine the patient's eligibility for one of the corresponding targeted anti-cancer therapies that have been approved for treatment.

Whole slide pathology images are high-resolution (e.g., megapixel or gigapixel) images of, e.g., tissue specimens that have been excised during biopsy or surgical procedures and subsequently analyzed for, e.g., diagnostic and/or curative intent. The ability to determine altered gene states in tissue samples directly from pathology slide images would provide the potential to vastly improve turnaround-times for patients and healthcare providers in the case of identifying the presence of certain key actionable gene alteration states (e.g., detection of EGFR/ALK mutations in non-small-cell lung carcinoma (NSCLC) tissue samples, or detection of gene fusions/rearrangements in lung adenocarcinoma tissue samples) to guide the choice of treatment by chemotherapy or a targeted anti-cancer therapy.

Previously reported methods for predicting gene alterations from whole slide pathology images rely on training a machine learning model using annotated pathology slide images of highly selected tissue samples that are fairly homogeneous in terms of tissue phenotype (e.g., tissue morphology or tissue histology). See, for example, Coudray, et al. 2018, “Classification and Mutation Prediction from Non-Small Cell Lung Cancer Histopathology Images Using Deep Learning”, Nature Medicine 24:1559-1567. These methods do not perform well when using pathology images of heterogeneous real-world tissue samples for which paired normal tissue samples are unavailable. Accordingly, there is a need for improved methods and systems for predicting gene alteration states from pathology slide images with greater accuracy and efficiency.

### BRIEF SUMMARY OF THE INVENTION

Described herein are methods and systems for more accurately determining gene alteration states in heterogeneous, real-world samples, e.g., tissue samples, using a machine learning-based analysis of pathology images. The disclosed methods rely on identifying tumor and non-tumor regions within the whole slide image based on labeled image patch-level data, identifying a set of buffer region (non-tumor) image patches based on the identified tumor region(s) and a proximity relationship, and providing tumor region image patches and buffer (non-tumor) region image patches as input to a trained gene alteration state classification model configured to predict the presence of at least on gene alteration state based on image features identified in the tumor and buffer region image patches. In some instances, the method may further comprise generating the labeled image patches by inputting a plurality of image patches derived from the pathology whole slide image into a trained tissue phenotype classification model, where the tissue phenotype classification model is configured to classify each image patch and output a tissue phenotype classification label for the image patch. By sampling both tumor region and buffer region image patches, the disclosed methods can leverage complex tissue interactions/relationships (e.g., intra- and peri-tumoral relationships that may be lost when only sampling tumor region image patches or when randomly sampling image patches across all tissue types visible in a pathology whole slide image) to improve the accuracy of predicting gene alterations present in tumor tissue.

Disclosed herein are methods for predicting a gene alteration state in a sample, the methods comprising: receiving, at one or more processors, a plurality of image patches derived from at least one slide image of the sample, wherein each image patch of the plurality is associated with a tissue phenotype classification label; identifying, using the one or more processors, a set of tumor region image patches from the plurality of image patches based on the tissue phenotype classification label for each image patch of the plurality of image patches; identifying, using the one or more processors, a set of buffer region image patches from the plurality of image patches based on the identified set of tumor region patches and a set of proximity criteria used to define buffer region image patches; inputting, using the one or more processors, the set of tumor region image patches and the set of buffer region image patches into a gene alteration state classification model to obtain a prediction of a presence of at least one gene alteration state for the tissue sample; and outputting, using the one or more processors, the prediction of a presence of the at least one gene alteration state for the sample.

In some embodiments, |the method further comprises generating the plurality of image patches by inputting, using the one or more processors, the plurality of image patches into a tissue phenotype classification model, wherein the tissue phenotype classification model is configured to classify each image patch and output a tissue phenotype classification label for the image patch.

In some embodiments, identifying the set of tumor region image patches comprises applying a binary mask based on tissue phenotype classification label to the plurality of image patches.

In some embodiments, the set of proximity criteria comprises an adjacency criterion, a distance criterion, or any combination thereof. In some embodiments, identifying the set of buffer region image patches based on the identified set of tumor region patches and the set of proximity criteria comprises identifying a set of n nearest neighboring, non-tumor region image patches adjacent to a tumor region indicated by the identified set of tumor region patches. In some embodiments, n is greater than or equal to 8, 10, 12, 14, 16, 18, 20, 40, 60, 80, 100, 120, 140, 160, 180, or 200 non-tumor region image patches.

In some embodiments, identifying the set of buffer region image patches based on the identified set of tumor region patches and the set of proximity criteria comprises identifying a set of n neighboring, non-tumor region image patches that are within a specified number of image pixels, m, of an edge of a tumor region indicated by the identified set of tumor region patches. In some embodiments, m is greater than or equal to 1, 2, 3, 4, 5, 6, 7, 8, 9, 10, 20, 30, 40, 50, 60, 70, 80, 90, or 100 image pixels.

In some embodiments, identifying the set of buffer region image patches based on the identified set of tumor region patches and the set of proximity criteria comprises identifying a set of n neighboring, non-tumor region image patches that are within a predefined distance, d, of an edge of a tumor region indicated by the identified set of tumor region patches. In some embodiments, d is greater than or equal to 1, 2, 3, 4, 5, 6, 7, 8, 9, 10, 20, 30, 40, 50, 60, 70, 80, 90, or 100 micrometers.

In some embodiments, the tissue phenotype classification model comprises a supervised machine learning model. In some embodiments, the supervised machine learning model comprises a neural network. In some embodiments, the neural network model comprises a convolutional neural network.

In some embodiments, the tissue phenotype classification model is trained on a plurality of annotated image patches (training image patches) derived from a plurality of slide images of samples from a cohort of subjects diagnosed with a disease of interest. In some embodiments, the disease of interest is a cancer.

In some embodiments, the tissue phenotype classification label corresponds to one of a plurality of tissue phenotypes comprising a tumor tissue phenotype, a normal tissue phenotype, a necrotic tissue phenotype, a stromal tissue phenotype, an immune tissue phenotype, or any combination thereof.

In some embodiments, the gene alteration state classification model comprises a supervised machine learning model. In some embodiments, the supervised machine learning model comprises a neural network. In some embodiments, the neural network model comprises a convolutional neural network.

In some embodiments, the gene alteration state classification model is trained on tumor region and buffer region image patches derived from slide images and associated gene alteration state data for a plurality of samples from a cohort of subjects diagnosed with a disease of interest. In some embodiments, the disease of interest is a cancer. In some embodiments, the associated gene alteration state data comprises nucleic acid sequence data that indicates a presence of at least one gene alteration state in the samples from the cohort of subjects. In some embodiments, the nucleic acid sequence data is derived from a next generation sequencing method. In some embodiments, the next generation sequencing method comprises a targeted sequencing method.

In some embodiments, the prediction of at least one gene alteration state for the sample comprises a prediction of a presence of at least 1, 2, 3, 4, 5, 6, 7, 8, 9, or 10 gene alterations in the sample. In some embodiments, the prediction of at least one gene alteration state comprises a prediction of a presence of at least 1, 2, 3, 4, 5, 6, 7, 8, 9, or 10 driver mutations in the sample.

In some embodiments, the prediction of at least one gene alteration state for the sample comprises a prediction of a presence of an EGFR alteration, ALK alteration, ROS1 alteration, NTRK1 alteration, NTRK2 alteration, NTRK3 alteration, KRAS alteration, G12C alteration, or any combination thereof in the sample.

In some embodiments, the method further comprises generating the image patches of the plurality by processing the at least one slide image of the sample using an image segmentation algorithm. In some embodiments, each image patch of the plurality of image patches is of a uniform, predetermined shape and size. In some embodiments, the uniform, predetermined shape is square or rectangular. In some embodiments, the uniform, predetermined size ranges from 32 pixels to 2048 pixels in at least one dimension.

In some embodiments, the at least one slide image of the sample comprises 1, 2, 3, 4, 5, 6, 7, 8, 9, or 10 slide images of the sample. In some embodiments, the at least one slide image of the sample comprises a pathology slide image of the sample.

In some embodiments, the sample comprises a tissue sample. In some embodiments, the tissue sample comprises a tissue biopsy sample. In some embodiments, the tissue sample comprises a needle core biopsy sample.

Also disclosed herein are systems comprising: one or more processors; and a memory communicatively coupled to the one or more processors and configured to store instructions that, when executed by the one or more processors, cause the system to perform any of the methods described herein.

Also disclosed herein are non-transitory computer-readable storage media storing one or more programs, the one or more programs comprising instructions, which when executed by one or more processors of a system, cause the system to perform any of the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for predicting a gene alteration state in a sample based on an analysis of a slide image of the sample according to one implementation of the disclosed methods.

FIG. 2 provides another non-limiting example of a process flowchart for predicting a gene alteration state in a sample based on an analysis of a slide image of the sample according to another implementation of the disclosed methods.

FIG. 3 provides a non-limiting example of a process flowchart for training a gene alteration state classifier.

FIG. 4 provides a non-limiting schematic illustration of a slide image comprising tumor and non-tumor tissue regions and associated image patches.

FIG. 5 depicts an exemplary computing device or system in accordance with one embodiment of the present disclosure.

FIG. 6 depicts an exemplary computer system or computer network, in accordance with some instances of the systems described herein.

### DETAILED DESCRIPTION

Methods and systems for more accurately determining gene alteration states in heterogeneous, real-world samples, e.g., tissue samples, using a machine learning-based analysis of pathology images are described. The disclosed methods rely on identifying tumor and non-tumor regions within the whole slide image based on labeled image patch-level data, identifying a set of buffer region (non-tumor) image patches based on the identified tumor region(s) and a proximity relationship, and providing tumor region image patches and buffer (non-tumor) region image patches as input to a trained gene alteration state classification model configured to predict the presence of at least on gene alteration state based on image features identified in the tumor and buffer region image patches. In some instances, the method may further comprise generating the labeled image patches by inputting a plurality of image patches derived from the pathology whole slide image into a trained tissue phenotype classification model, where the tissue phenotype classification model is configured to classify each image patch and output a tissue phenotype classification label for the image patch. By sampling both tumor region and buffer region image patches, the disclosed methods can leverage complex tissue interactions/relationships (e.g., intra- and peri-tumoral relationships that may be lost when only sampling tumor region image patches or when randomly sampling image patches across all tissue types visible in a pathology whole slide image) to improve the accuracy of predicting gene alterations present in tumor tissue.

In some instances, for example, methods for predicting a gene alteration state in a sample are described that comprise receiving a plurality of image patches derived from at least one slide image of the sample (e.g., a tissue sample), where each image patch of the plurality is associated with a tissue phenotype classification label; identifying a set of tumor region image patches from the plurality of image patches based on the tissue phenotype classification label for each image patch of the plurality of image patches; identifying a set of buffer region image patches from the plurality of image patches based on the identified set of tumor region patches and a set of proximity criteria used to define buffer region image patches; inputting the set of tumor region image patches and the set of buffer region image patches into a gene alteration state classification model configured to predict a presence of at least one gene alteration state based on image features identified in the sets of tumor region and buffer region image patches; and outputting a prediction of the presence of the at least one gene alteration state for the sample.

In some instances, the method may further comprise generating the plurality of labeled image patches by inputting a plurality of image patches into a tissue phenotype classification model, where the tissue phenotype classification model is configured to classify each image patch and output a tissue phenotype classification label for the image patch.

In some instances, the set of proximity criteria may comprise an adjacency criterion, a distance criterion, or any combination thereof.

In some instances, identifying the set of buffer region image patches based on the identified set of tumor region patches and the set of proximity criteria may comprise, for example, identifying a set of n nearest neighboring, non-tumor region image patches adjacent to a tumor region indicated by the identified set of tumor region patches.

In some instances, identifying the set of buffer region image patches based on the identified set of tumor region patches and the set of proximity criteria may comprise, for example, identifying a set of n neighboring, non-tumor region image patches that are within a specified number of image pixels, m, of an edge of a tumor region indicated by the identified set of tumor region patches.

In some instances, identifying the set of buffer region image patches based on the identified set of tumor region patches and the set of proximity criteria may comprise, for example, identifying a set of n neighboring, non-tumor region image patches that are within a predefined distance, d, of an edge of a tumor region indicated by the identified set of tumor region patches.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the term “sequence read” can refer to a raw sequence read (e.g., a sequence read as obtained directly from a sequencing instrument), an aligned sequence read (e.g., a sequence read that has been aligned to a reference genome), a single-end sequence read, a paired-end sequence read, a merged sequence read (e.g., a sequence read based on merging a group of overlapping paired-end reads), a consensus sequence read (e.g., a sequence read based on performing error correction on a merged sequence read), a computationally reconstructed sequence read (e.g., a sequence read that has been computationally truncated at the 5′ and/or 3′ end), or any combination thereof.

As used herein, the terms “variant sequence” or “variant” are used interchangeably and refer to a modified nucleic acid sequence relative to a corresponding “normal” or “wild-type” sequence.

In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

Methods for Machine Learning-Based Prediction of Gene Alterations from Pathology Images

The disclosed methods utilize a machine learning approach (e.g., a supervised learning or a Multiple Instance Learning (MIL) approach) to train a gene alteration state classifier for prediction of whether or not a gene alteration (e.g., an EGFR mutation) is present in a sample (e.g., a tissue sample) from digital pathology images. In some instances, the gene alteration state classifier may be used in combination with an upstream trained tissue morphology classifier (e.g., a convolutional neural network trained to cluster image patches into different tissue morphology classes commonly found in histopathology slide images (such as tumor, normal, immune, stroma, and necrosis) to process image patches extracted from digital pathology slides and predict the presence or absence of a gene alteration state in tissue specimens or other samples depicted in the digital pathology images. The motivation underlying the development of the disclosed methods is to better capture the intra-tumoral and peri-tumoral morphological relationships between tumor regions and neighboring non-tumor regions of a tissue sample (e.g., immune, stromal, necrotic, and/or normal tissue regions of the tissue sample) within randomly or non-randomly selected sets of whole slide image patches.

In brief, a cohort of relevant pathology slide images of, e.g., lung tissue specimens collected from patients with lung adenocarcinoma (along with associated features, such as disease ontology, tissue sampling site, and specimen type (e.g., biopsy specimen versus surgical resection specimen)) can be constructed for analysis. The tissue morphology classifier can be trained on a random subset of these slides (e.g., following annotation by a trained pathologist), and can then be used to predict relevant tissue profiles across all slide images in the cohort. In some instances, the tissue classifier may comprise the use of binary masks that are constructed using image processing techniques to discriminate between tissue regions and non-tissue regions and/or to discriminate between tumor and non-tumor regions across all slide images. Image patches of predetermined size may then be extracted from the identified tissue and/or tumor/non-tumor regions.

The disclosed methods sample image patches within classified tumor patch regions along with image patches derived from an adjacent “buffer” region to capture intra-tumoral and peri-tumoral morphological relationships within neighboring tissue morphologies, e.g., immune and stromal tissue regions. The patch-level tissue morphology labels are thus used to selectively sample desired tissue regions from the whole slide images rather than taking a random subset of image patches that may include less informative or less disease-relevant tissue regions, such as normal tissue regions. This work was motivated by findings in an EGFR alteration prediction experiment which indicated that analysis of a heterogeneous set of image patches outperformed analyses based on sets of image patches comprising only tumor-region image patches. Furthermore, a post-hoc analysis showed a relationship between EGFR alteration predictions and inclusion of immune tissue region image patches. Thus, in some instances the disclosed method may analyze all or a portion of the image patches labeled as “tumor”, and a set of surrounding image patches chosen on the basis of a proximity relationship (e.g., all image patches present within n pixels of the tumor region, thereby creating a tunable “buffer” region where n may be adjusted). This approach may allow for a more theoretically informative set of image patches to be included in the training data set for the gene alteration classifier while maintaining neighboring tissue relationships like peri-tumoral and intra-tumoral immune interactions.

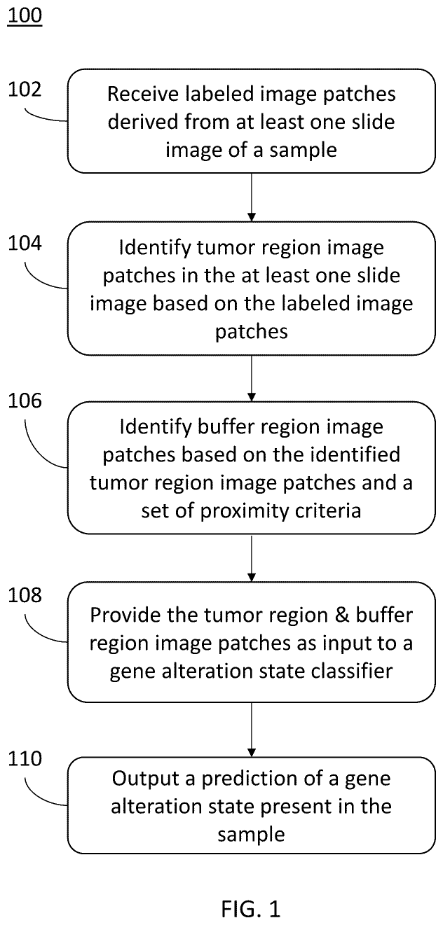

FIG. 1 provides a non-limiting example of a flowchart for a process 100 for predicting a gene alteration state in a tissue sample based on a slide image of the tissue sample. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, labeled image patches derived from at least one slide image of a sample are received, e.g., by the one or more processors of a system configured to execute the method illustrated in FIG. 1, where each image patch is associated with a tissue phenotype classification label.

In some instances, the sample may comprise a tissue sample, for example a tissue biopsy sample or a needle core biopsy sample.

In some instances, the at least one slide image of the sample may comprise a pathology slide image of the sample. In some instances, the at least one slide image of the sample may comprise 1, 2, 3, 4, 5, 6, 7, 8, 9, 10, or more than 10 slide images of the sample.

In some instances, the method may further comprise generating the image patches by processing the at least one slide image using, for example, an image segmentation algorithm such as a region-based, edge detection-based, or clustering based segmentation algorithm. In some instances, image patches may be generated by simply grouping image pixels according to a grid-based segmentation scheme. Any of a variety of image processing methods known to those of skill in the art may be used for slide image processing and/or pre-processing. Examples include, but are not limited to, Canny edge detection methods, Canny-Deriche edge detection methods, first-order gradient edge detection methods (e.g., the Sobel operator), second order differential edge detection methods, phase congruency (phase coherence) edge detection methods, other image segmentation algorithms (e.g., intensity thresholding, intensity clustering methods, intensity histogram-based methods, etc.), feature and pattern recognition algorithms (e.g., the generalized Hough transform for detecting arbitrary shapes, the circular Hough transform, etc.), and mathematical analysis algorithms (e.g., Fourier transform, fast Fourier transform, wavelet analysis, auto-correlation, etc.), or any combination thereof.

In some instances, each image patch of the plurality of image patches extracted from a slide image may be of a uniform, predetermined shape and/or size, for example, a square or rectangular shape. In some instances, each image patch may range in size from about 32 pixels to about 2048 pixels in at least one dimension. In some instances, for example, each image patch may be at least 32, 64, 128, 256, 512, 1024, or 2048 pixels in at least one dimension. In some instances, each image patch may be at most 2048, 1024, 512, 256, 128, 64, or 32 pixels in at least one dimension. Any of the lower and upper values described in this paragraph may be combined to form a range included within the present disclosure, for example, in some instances each image patch may range in size from about 128 to about 1024 pixels in at least one dimension. Those of skill in the art will recognize that the at least one dimension of each image patch may have any value within the range disclosed herein, e.g., 48 pixels.

In some instances, the method may further comprise generating the labeled image patches by processing the at least one slide image to extract a plurality of image patches, and having a trained pathologist annotate each image patch with a tissue phenotype classification label.

In some instances, the plurality of image patches may be input into a tissue phenotype classification model (see, e.g., International Patent Application Publication No. WO 2022/225995, which is incorporated herein by reference in its entirety) that is configured to classify each image patch and output a tissue phenotype classification label for the image patch. In some instances, for example, the tissue phenotype classification label may correspond to one of a plurality of tissue phenotypes comprising, for example, a tumor tissue phenotype, a normal tissue phenotype, a necrotic tissue phenotype, a stromal tissue phenotype, an immune tissue phenotype, or any combination thereof. In some instances, the tissue phenotype classification label may further comprise, e.g., a “no tissue present” label.

The tissue phenotype classification model may comprise, for example, a supervised machine learning model. In some instances, the supervised machine learning model may comprise a neural network, for example, a convolutional neural network, that is trained on a plurality of annotated image patches (training image patches) derived from a plurality of slide images of samples from a cohort of subjects diagnosed with a disease of interest (e.g., cancer).

At step 104 in FIG. 1, tumor region image patches are identified across the at least one slide image based on the labeled image patches. For example, a binary mask based on the tissue phenotype classification labels (e.g., a tumor tissue phenotype label versus any other tissue phenotype classification label) may be applied to the at least one slide image to distinguish between tumor region image patches and non-tumor region image patches.

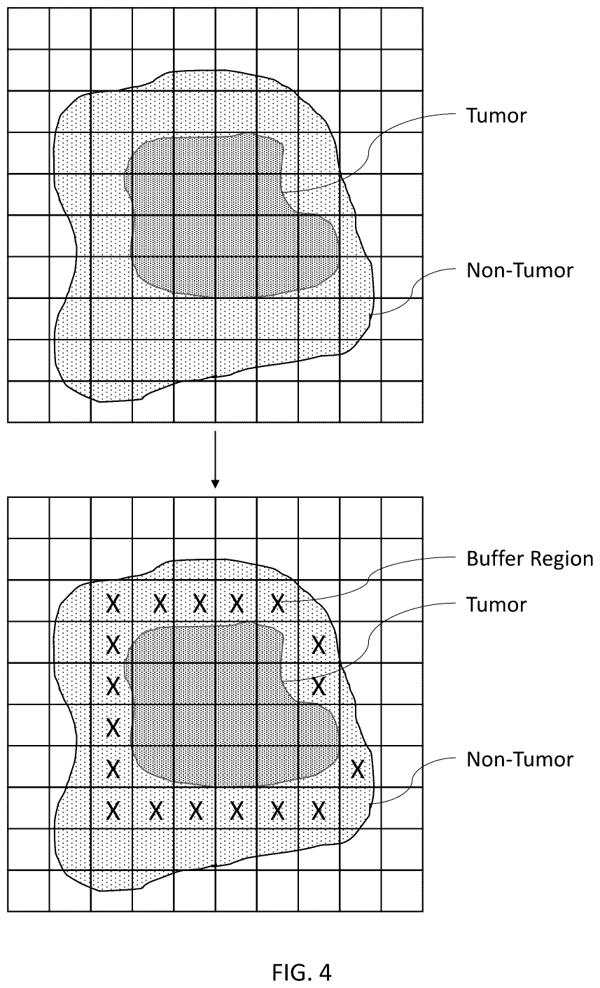

At step 106 in FIG. 1, buffer region image patches are identified based on the previously identified tumor region image patches and a set of proximity criteria that are used to define “buffer region” image patches. Buffer region image patches can be, for example, image patches that contact or are adjacent to an edge of a tumor region image patch, or that are within close proximity of a tumor region image patch. A non-limiting schematic illustration of tumor region image patches and buffer region image patches is provided in FIG. 4, which will be described in more detail below. The set of proximity criteria can comprise, for example, an adjacency criterion, a distance criterion, or any combination thereof.

In some instances, identifying the set of buffer region image patches based on the identified set of tumor region patches and the set of proximity criteria may comprise identifying a set of n nearest neighboring, non-tumor region image patches adjacent to a tumor region indicated by the identified set of tumor region patches. In some instances, for example, n may be greater than or equal to 8, 10, 12, 14, 16, 18, 20, 40, 60, 80, 100, 120, 140, 160, 180, or 200 non-tumor region image patches.

In some instances, identifying the set of buffer region image patches based on the identified set of tumor region patches and the set of proximity criteria may comprise identifying a set of n neighboring, non-tumor region image patches that are within a specified number of image pixels, m, of an edge of a tumor region indicated by the identified set of tumor region patches. In some instances, for example, m may be greater than or equal to 1, 2, 3, 4, 5, 6, 7, 8, 9, 10, 20, 30, 40, 50, 60, 70, 80, 90, or 100 image pixels.

In some instances, identifying the set of buffer region image patches based on the identified set of tumor region patches and the set of proximity criteria may comprise identifying a set of n neighboring, non-tumor region image patches that are within a predefined distance, d, of an edge of a tumor region indicated by the identified set of tumor region patches. In some instances, for example, d may be greater than or equal to 1, 2, 3, 4, 5, 6, 7, 8, 9, 10, 20, 30, 40, 50, 60, 70, 80, 90, or 100 micrometers.

At step 108 in FIG. 1, the tumor region and buffer region image patches are provided as input to a gene alteration state classifier.

Any of a variety of machine learning approaches & algorithms (where a machine learning model, as referred to herein, comprises a trained machine learning algorithm) may be used in implementing the disclosed methods. In some instances, for example, the gene alteration state classification model may comprise a supervised, semi-supervised, weakly-supervised, or unsupervised machine learning model. In some instances, the gene alteration state classification model may comprise a supervised machine learning model such as a neural network (e.g., a convolutional neural network) or deep learning model. In some instances, the gene alteration state classification model may comprise a neural network (e.g., a convolutional neural network) trained using a Multiple Instance Learning (MIL)-based approach.

Neural networks and deep learning algorithms are algorithms inspired by the structure and function of the human brain. Specifically, deep learning algorithms are large neural networks comprising many layers of coupled “nodes” that may be used to map input feature data to, e.g., classification decisions. In some instances, the machine learning model/algorithm used for implementing the disclosed methods and systems may be a neural network or deep learning model/algorithm that comprises any type of neural network model known to those of skill in the art, such as a feedforward neural network, radial basis function network, recurrent neural network, or convolutional neural network, and the like. In some instances, the disclosed methods and systems may employ a pre-trained neural network or deep learning model. In some embodiment, the disclosed methods and systems may employ a continuous learning neural network or deep learning model, where the model is periodically or continuously updated based on new training data provided by, e.g., a single local operational system, a plurality of local operational systems, or a plurality of geographically-distributed operational systems.

Neural networks generally comprise an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to a preferred output value or set of output values. Each layer of the neural network comprises a number of nodes (or neurons). A node receives input data (e.g., image feature data derived from image patch data, or other types of input data) that comes either directly from the input data nodes or from the output of nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a neuron may be gated using a threshold or activation function, f, which may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, a sigmoid function, or any combination thereof.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., a prediction of a gene alteration state) that the neural network predicts are consistent with the examples included in the training data set. The adjustable parameters of the model may be obtained from, e.g., a back propagation neural network training process that may or may not be performed using the same hardware as that used for processing images and/or predicting gene alteration states.

Other specific types of deep machine learning models/algorithms, e.g., convolutional neural networks (CNNs) (often used for the processing of image data from machine vision systems) may also be used by the disclosed methods and systems. CNNs are commonly composed of layers of different types: convolution, pooling, upscaling, and fully-connected node layers. In some cases, an activation function such as rectified linear unit may be used in some of the layers. In a CNN architecture, there can be one or more layers for each type of operation performed. A CNN architecture may comprise any number of layers in total, and any number of layers for the different types of operations performed. The simplest convolutional neural network architecture starts with an input layer followed by a sequence of convolutional layers and pooling layers, and ends with fully-connected layers. Each convolution layer may comprise a plurality of parameters used for performing the convolution operations. Each convolution layer may also comprise one or more filters, which in turn may comprise one or more weighting factors or other adjustable parameters. In some instances, the parameters may include biases (i.e., parameters that permit the activation function to be shifted). In some cases, the convolutional layers are followed by a layer of ReLU activation function. Other activation functions can also be used, for example the saturating hyperbolic tangent, identity, binary step, logistic, arcTan, softsign, parameteric rectified linear unit, exponential linear unit, softPlus, bent identity, softExponential, Sinusoid, Sine, Gaussian, the sigmoid function and various others. The convolutional, pooling and ReLU layers may function as learnable features extractors, while the fully connected layers may function as a machine learning classifier. As with other neural networks, the convolutional layers and fully-connected layers of CNN architectures typically include various adjustable computational parameters, e.g., weights, bias values, and threshold values, that are trained in a training phase as described above.

Supervised learning models/algorithms: Supervised learning models are trained algorithms that rely on the use of labeled training data to infer the relationship between a set of one or more input features (e.g., tumor region image patches, buffer region image patches, or image features derived therefrom) and an output prediction (e.g., a prediction of the presence of a gene alteration in a tissue sample). The training data comprises one or more sets (e.g., 1, 2, 3, 4, 5, 6, 7, 8, 9, 10, or more than 10 sets) of paired training examples, where each example comprises a set of input features (e.g., tumor region image patches, buffer region image patches, or image features derived therefrom) for a given sample and a “ground truth” classification of the sample as comprising a given gene alteration.

Multiple instance learning models/algorithms: Multiple Instance Learning (MIL) is a form of weakly supervised learning where training data examples are arranged in sets, called bags, and a label is provided for the entire bag (see, e.g., Carbonneau, et al. (2016) “Multiple Instance Learning: A Survey of Problem Characteristics and Applications”, arXiv:1612.03365). Weakly-supervised methods, such as MIL, can alleviate the burden of compiling the large quantities of labeled training data required for addressing large-scale problems since weak supervision is generally obtained more efficiently.

In some instances, MIL models may further comprise an attention mechanism to increase model performance (e.g., accuracy) by focusing on the most relevant aspects of the input data and giving more weight to important features. For example, the features (embeddings) from the hidden layer preceding the attention mechanism can be considered by the model, via the attention mechanism, to determine the select input instances that are most important for accurate prediction. The attention mechanism will generate a relative importance value for each embedding corresponding to each input instance which is then used to compute a weighted average of feature values. The weighted average of the feature (embedding) values is then passed into a classification layer(s) for the final slide-level prediction.

Semi-supervised learning models/algorithms: Semi-supervised learning models are trained algorithms that make use of both labeled and unlabeled data for training (typically using a relatively small amount of labeled data with a larger amount of unlabeled data).

Unsupervised learning models/algorithms: Unsupervised learning models are trained algorithms used to draw inferences from training datasets consisting of, e.g., image patch datasets that are not paired with labeled gene alteration state classification data. One example of a commonly used unsupervised learning algorithm is cluster analysis, which is often used for exploratory data analysis to find hidden patterns or groupings in multi-dimensional data sets. Other examples of unsupervised learning algorithms include, but are not limited to, neural networks, association rule learning algorithms, hierarchical clustering algorithms, matrix factorization approaches, dimensionality reduction approaches, or any combination thereof.

In some instances, the gene alteration state classification model may be trained on tumor region and buffer region image patches derived from slide images and associated gene alteration state data for a plurality of samples from a cohort of subjects diagnosed with a disease of interest, e.g., a cancer. The associated gene alteration state data may comprise, for example, microarray-based genotyping data or nucleic acid sequence data that indicates a presence of at least one gene alteration state in the samples from the cohort of subjects. In some instances, the nucleic acid sequence data may be derived from a next generation sequencing method, e.g., a whole genome sequencing method, a whole exome sequencing method, or a targeted sequencing method.

At step 110 in FIG. 1, a prediction of whether or not at least one gene alteration state is present in the sample is output. In some instances, the prediction of at least one gene alteration state for the sample may comprise a prediction of a presence of at least 1, 2, 3, 4, 5, 6, 7, 8, 9, or 10 gene alterations in the sample. In some instances, the prediction of at least one gene alteration state may comprise a prediction of a presence of at least 1, 2, 3, 4, 5, 6, 7, 8, 9, or 10 driver mutations in the sample.

In some instances, for example, the prediction of at least one gene alteration state for the sample comprises a prediction of a presence of an EGFR alteration, ALK alteration, ROS1 alteration, NTRK1 alteration, NTRK2 alteration, NTRK3 alteration, KRAS alteration, G12C alteration, or any combination thereof.

The disclosed methods may potentially be used for, e.g., pre-screening for actionable gene alterations evident in digital pathology images and/or for future treatment outcome-related methods that would benefit from selective region-sampling of image patches. If clinical grade performance specifications are met, then off-site pathology labs could upload scanned images of locally procured specimens to a server. The disclosed methods, deployed in the cloud, could then be used to predict gene alteration status to provide enhanced decision-making insights prior to receipt of the physical sample at a genomic profiling laboratory for actual sequencing.

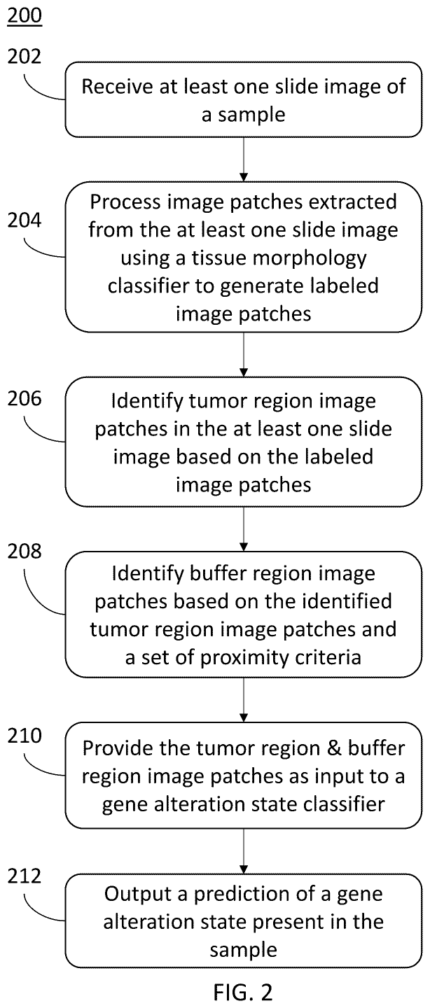

FIG. 2 provides another non-limiting example of a flowchart for a process 200 for predicting a gene alteration state in a tissue sample based on a slide image of the tissue sample. Process 200 can be performed, for example, using one or more electronic devices implementing a software platform. In some examples, process 200 is performed using a client-server system, and the blocks of process 200 are divided up in any manner between the server and a client device. In other examples, the blocks of process 200 are divided up between the server and multiple client devices. Thus, while portions of process 200 are described herein as being performed by particular devices of a client-server system, it will be appreciated that process 200 is not so limited. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

At step 202 in FIG. 2, at least one slide image of a sample, e.g., by the one or more processors of a system configured to execute the method illustrated in FIG. 2.

As noted above with regard to the process illustrated in FIG. 1, the sample may comprise, for example, a tissue sample, for example a tissue biopsy sample or a needle core biopsy sample.

In some instances, the at least one slide image of the sample may comprise a pathology slide image of the sample. In some instances, the at least one slide image of the sample may comprise 1, 2, 3, 4, 5, 6, 7, 8, 9, 10, or more than 10 slide images of the sample.

At step 204 in FIG. 2, image patches extracted from the at least one slide image are processed using a tissue morphology classifier to generate labeled image patches.

Image patches having properties such as those described above in reference to FIG. 1 may be generated by processing the at least one slide image using any of a variety of image segmentation algorithms known to those of skill in the art, such as a region-based, edge detection-based, or clustering based segmentation algorithms. In some instances, image patches may be generated by simply grouping image pixels according to a grid-based segmentation scheme. Any of a variety of image processing methods known to those of skill in the art, as described above in more detail in reference to FIG. 1, may be used for slide image processing and/or pre-processing

The image patches extracted from the at least one slide image may be input into a tissue phenotype classification model that is configured to classify each image patch and output a tissue phenotype classification label for the image patch. In some instances, for example, the tissue phenotype classification label may correspond to one of a plurality of tissue phenotypes comprising a tumor tissue phenotype, a normal tissue phenotype, a necrotic tissue phenotype, a stromal tissue phenotype, an immune tissue phenotype, or any combination thereof. In some instances, the tissue phenotype classification label may further comprise, e.g., a “no tissue present” label.

The tissue phenotype classification model may comprise, for example, a supervised machine learning model. In some instances, the supervised machine learning model may comprise a neural network, for example, a convolutional neural network, that is trained on a plurality of annotated image patches (training image patches) derived from a plurality of slide images of samples from a cohort of subjects diagnosed with a disease of interest (e.g., cancer).

At step 206 in FIG. 2, tumor region image patches are identified in the at least one slide image based on the labeled image patches. For example, a binary mask based on the tissue phenotype classification labels (e.g., a tumor tissue phenotype label versus any other tissue phenotype classification label) may be applied to the at least one slide image to distinguish between tumor region image patches and non-tumor region image patches.

At step 208 in FIG. 2, buffer region image patches are identified based on the previously identified tumor region image patches and a set of proximity criteria that are used to define “buffer region” image patches. Buffer region image patches can be, for example, image patches that contact or are adjacent to an edge of a tumor region image patch, or that are within close proximity of a tumor region image patch. As noted above, a non-limiting schematic illustration of tumor region image patches and buffer region image patches is provided in FIG. 4, which will be described in more detail below. The set of proximity criteria can comprise, for example, an adjacency criterion, a distance criterion, or any combination thereof.

In some instances, identifying the set of buffer region image patches based on the identified set of tumor region patches and the set of proximity criteria may comprise identifying a set of n nearest neighboring, non-tumor region image patches adjacent to a tumor region indicated by the identified set of tumor region patches. In some instances, for example, n may be greater than or equal to 8, 10, 12, 14, 16, 18, 20, 40, 60, 80, 100, 120, 140, 160, 180, or 200 non-tumor region image patches.

In some instances, identifying the set of buffer region image patches based on the identified set of tumor region patches and the set of proximity criteria may comprise identifying a set of n neighboring, non-tumor region image patches that are within a specified number of image pixels, m, of an edge of a tumor region indicated by the identified set of tumor region patches. In some instances, for example, m may be greater than or equal to 1, 2, 3, 4, 5, 6, 7, 8, 9, 10, 20, 30, 40, 50, 60, 70, 80, 90, or 100 image pixels.

In some instances, identifying the set of buffer region image patches based on the identified set of tumor region patches and the set of proximity criteria may comprise identifying a set of n neighboring, non-tumor region image patches that are within a predefined distance, d, of an edge of a tumor region indicated by the identified set of tumor region patches. In some instances, for example, d may be greater than or equal to 1, 2, 3, 4, 5, 6, 7, 8, 9, 10, 20, 30, 40, 50, 60, 70, 80, 90, or 100 micrometers.

At step 210 in FIG. 2, the tumor region & buffer region image patches are provided as input to a gene alteration state classifier. Any of a variety of machine learning approaches & algorithms (where a machine learning model, as referred to herein, comprises a trained machine learning algorithm) may be used in implementing the disclosed methods. As noted above, in some instances the gene alteration state classification model may comprise a supervised, semi-supervised, weakly-supervised, or unsupervised machine learning model. In some instances, the gene alteration state classification model may comprise a supervised machine learning model such as a neural network (e.g., a convolutional neural network) or deep learning model. In some instances, the gene alteration state classification model may comprise a neural network (e.g., a convolutional neural network) trained using a Multiple Instance Learning (MIL)-based approach.

At step 212 in FIG. 2, a prediction of whether or not at least one gene alteration state is present in the sample is output. In some instances, the prediction of at least one gene alteration state for the sample may comprise a prediction of a presence of at least 1, 2, 3, 4, 5, 6, 7, 8, 9, or 10 gene alterations in the sample. In some instances, the prediction of at least one gene alteration state may comprise a prediction of a presence of at least 1, 2, 3, 4, 5, 6, 7, 8, 9, or 10 driver mutations in the sample.

In some instances, for example, the prediction of at least one gene alteration state for the sample comprises a prediction of a presence of an EGFR alteration, ALK alteration, ROS1 alteration, NTRK1 alteration, NTRK2 alteration, NTRK3 alteration, KRAS alteration, G12C alteration, or any combination thereof.

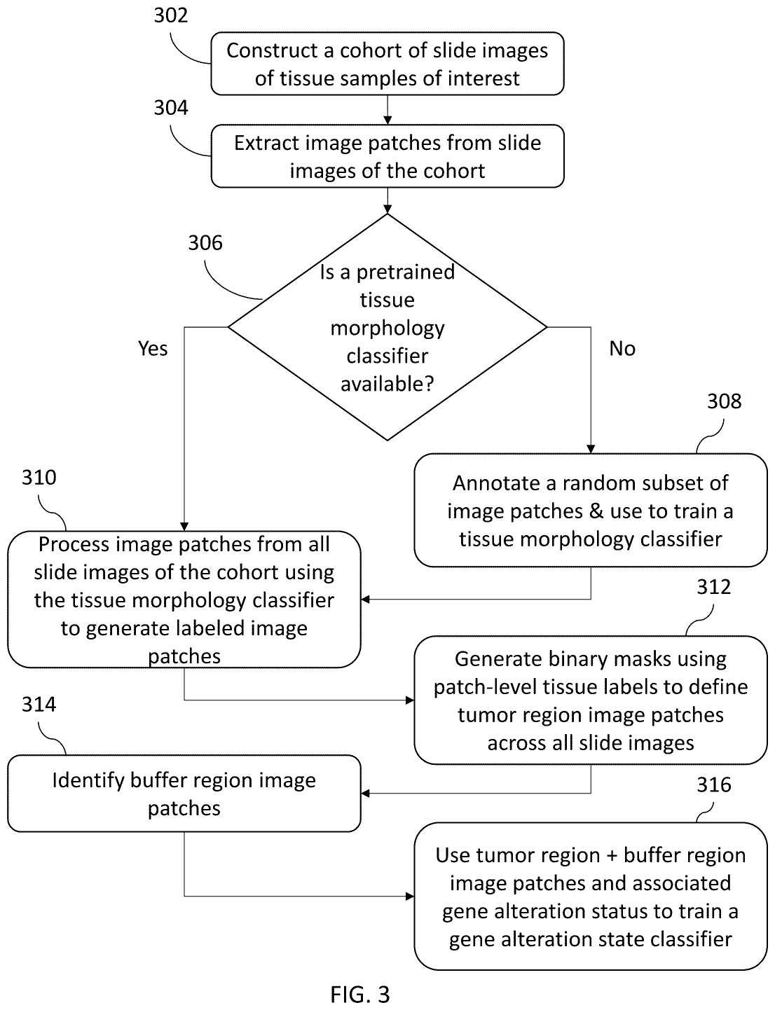

FIG. 3 provides a non-limiting example of a flowchart for a process 300 for training a gene alteration state classifier. Process 300 can be performed, for example, using one or more electronic devices implementing a software platform. In some examples, process 300 is performed using a client-server system, and the blocks of process 300 are divided up in any manner between the server and a client device. In other examples, the blocks of process 300 are divided up between the server and multiple client devices. Thus, while portions of process 300 are described herein as being performed by particular devices of a client-server system, it will be appreciated that process 300 is not so limited. In other examples, process 300 is performed using only a client device or only multiple client devices. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail below) are exemplary by nature and, as such, should not be viewed as limiting.

At step 302 in FIG. 3, a cohort of slide images of samples (e.g., tissue samples) of interest is constructed. In some instances, for example, the samples of interest may comprise tissue samples from a cohort of patients diagnosed with a specific disease, e.g., a specific cancer. In some instances, the tissue samples may comprise, e.g., surgical resection samples or biopsy samples.

At step 304 in FIG. 3, image patches are extracted from the slide images of the cohort using, e.g., any of the techniques described above with respect to FIG. 1 and FIG. 2.

At step 306 in FIG. 3, a determination is made as to whether a pretrained tissue morphology classifier available. If not, the process proceeds to step 308. If a pretrained tissue morphology classifier is available, the process proceeds to step 310.

At step 308 in FIG. 3, a random subset of image patches from the cohort of slide images are annotated and used to train a tissue morphology classifier, which may then be used in the implementation of step 310.

At step 310 in FIG. 3, all of the image patches from the cohort of slide images are processed using the tissue morphology classifier to generate labeled image patches, i.e., image patches labeled with a tissue phenotype classification label. In some instances, for example, the tissue phenotype classification label may correspond to one of a plurality of tissue phenotypes comprising a tumor tissue phenotype, a normal tissue phenotype, a necrotic tissue phenotype, a stromal tissue phenotype, an immune tissue phenotype, or any combination thereof. In some instances, the tissue phenotype classification label may further comprise, e.g., a “no tissue present” label.

At step 312 in FIG. 3, binary masks are generated using the patch-level tissue labels to define tumor region image patches across all slide images of the cohort.

At step 314 in FIG. 3, buffer region image patches are identified for all slide images of the cohort based on, e.g., the previously identified tumor region image patches and a set of proximity criteria such as those described above with respect to FIG. 1 and FIG. 2.

At step 316 in FIG. 3, the tumor region image patches and buffer region image patches are used along with associated gene alteration status (determined using, e.g., microarray-based genotyping and/or next generation sequencing of nucleic acid molecules extracted from the cohort of samples) to train a gene alteration state classifier. The gene alteration state classifier may be configures to output a prediction of whether or not one or more gene alterations are present in the sample. For example, the prediction may comprise a prediction of a presence of at least 1, 2, 3, 4, 5, 6, 7, 8, 9, or 10 gene alterations in the sample. In some instances, the prediction may comprise a prediction of a presence of at least 1, 2, 3, 4, 5, 6, 7, 8, 9, or 10 driver mutations in the sample.

In some instances, the prediction may comprise a prediction of whether or not an EGFR alteration, ALK alteration, ROS1 alteration, NTRK1 alteration, NTRK2 alteration, NTRK3 alteration, KRAS alteration, G12C alteration, or any combination thereof, is present in the sample.

FIG. 4 provides a non-limiting example of a slide image comprising tumor and non-tumor tissue regions and associated image patches. In this schematic illustration, tumor tissue is identified by dark gray shading, and non-tumor tissue is identified by light gray shading. The grid represents a plurality of image patches extracted from the whole slide image using any of the techniques described above.

The image patches shown in FIG. 4 that are entirely or primarily dark gray provide examples of tumor region image patches. In some instances, image patches that comprise at least 50%, 60%, 70%, 80%, 85%, 90%, 95%, or 98% tumor tissue by area may be categorized as tumor region image patches.

The image patches shown in FIG. 4 that have been designated with an “X” provide non-limiting examples of buffer region image patches. As described above, buffer region image patches may be defined on the basis of the identified tumor region image patches and a specified set of proximity criteria, e.g., an adjacency criterion, a distance criterion (e.g., specified in terms of a number of microns or other actual distance unit, a number of image pixels, a number of intervening image patches, etc.), or any combination thereof.

In some instances, the disclosed methods may be used to analyze pathology images and identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to analyze pathology images and identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise performing sequencing of nucleic acid molecules extracted from the tissue sample(s) for which pathology slide images are analyzed, e.g., as part of generating a training data set for the gene alteration state classifier, or as part of confirming a prediction by the gene alteration state classifier. Thus, in some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for predicting a gene alteration state in a pathology image of a tissue sample may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for predicting a gene alteration state in a pathology image of a tissue sample may be used to select a subject (e.g., a patient) for a clinical trial based on the identification of a gene alteration for one or more gene loci. In some instances, patient selection for clinical trials based on, e.g., identification of a gene alteration at one or more gene loci, may accelerate the development of targeted therapies and improve the healthcare outcomes for treatment decisions.

In some instances, the disclosed methods for predicting a gene alteration state in a pathology image of a tissue sample may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy. surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for predicting a gene alteration state in a pathology image of a tissue sample may be used in treating a disease (e.g., a cancer) in a subject. For example, in response to identifying a gene alteration using any of the methods disclosed herein, an effective amount of an anti-cancer therapy or anti-cancer treatment may be administered to the subject.

In some instances, the disclosed methods for predicting a gene alteration state in a pathology image of a tissue sample may be used for monitoring disease progression or recurrence (e.g., cancer or tumor progression or recurrence) in a subject. For example, in some instances, the methods may be used to identify a gene alteration in a first sample obtained from the subject at a first time point, and used to identify a gene alteration in a second sample obtained from the subject at a second time point, where comparison of the first identification of a gene alteration and the second identification of a gene alteration allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the identification of a gene alteration.

In some instances, the prediction of a gene alteration in a tissue sample using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for predicting a gene alteration state in a pathology image of a tissue sample may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for predicting a gene alteration state in a pathology image of a tissue sample as part of a genomic profiling process can improve the validity of, e.g., disease detection calls and treatment decisions, made on the basis of the genomic profile by, for example, independently confirming the presence of a gene alteration in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythemato sus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or nonspecific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS platform. In some instances, sequencing may comprise Illumina MiSeq™ sequencing. In some instances, sequencing may comprise Illumina HiSeq® sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. ChT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Systems

Also disclosed herein are systems designed to implement any of the disclosed methods for predicting a gene alteration state based on analysis of a pathology image of a sample from a subject. The systems may comprise, e.g., one or more processors, and a memory unit communicatively coupled to the one or more processors and configured to store instructions that, when executed by the one or more processors, cause the system to: receive a plurality of image patches derived from at least one slide image of the sample (e.g., a tissue sample), where each image patch of the plurality is associated with a tissue phenotype classification label; identify a set of tumor region image patches from the plurality of image patches based on the tissue phenotype classification label for each image patch of the plurality of image patches; identify a set of buffer region image patches from the plurality of image patches based on the identified set of tumor region patches and a set of proximity criteria used to define buffer region image patches; input the set of tumor region image patches and the set of buffer region image patches into a gene alteration state classification model configured to predict a presence of at least one gene alteration state based on image features identified in the sets of tumor region and buffer region image patches; and output a prediction of the presence of the at least one gene alteration state for the sample.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for analysis of any of a variety of samples as described herein (e.g., a tissue sample, biopsy sample, hematological sample, or liquid biopsy sample derived from the subject).

In some instances, a plurality of gene loci for which sequencing data is processed to identify gene alterations may comprise at least 1, 2, 3, 4, 5, 6, 7, 8, 9, 10, 20, 30, 40, 50, 60, 70, 80, 0, 100, 200, 300, 400, 500, 600, 700, 800, 900, 1000, or more than 1000 gene loci (or any number of gene loci within the range of 1 to more than 1000 gene loci).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the disclosed methods for predicting a gene alteration based on an analysis of an image of a tissue sample may be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xi, and their associated weights, Wi. In some cases, the weighted sum is offset with a bias, b. In some cases, the output of a node may be gated using a threshold or activation function, f, where f may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

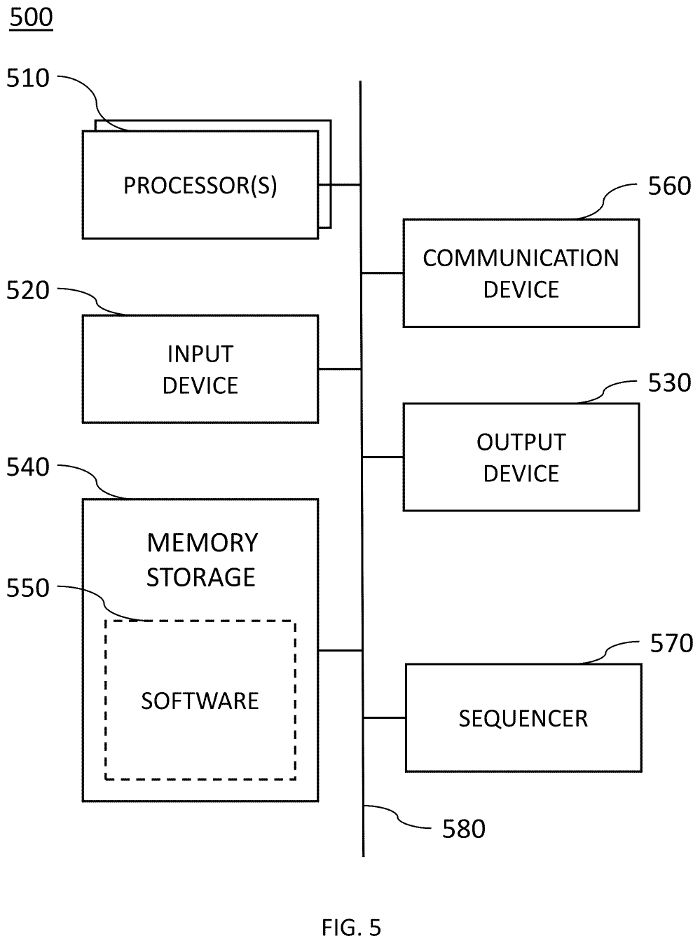

FIG. 5 illustrates an example of a computing device or system in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more processor(s) 510, input devices 520, output devices 530, memory or storage devices 540, communication devices 560, and nucleic acid sequencers 570. Software 550 residing in memory or storage device 540 may comprise, e.g., an operating system as well as software for executing the methods described herein. Input device 520 and output device 530 can generally correspond to those described herein, and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage (e.g., an electrical, magnetic or optical memory including a RAM (volatile and non-volatile), cache, hard drive, or removable storage disk). Communication device 560 can include any suitable device capable of transmitting and receiving signals over a network, such as a network interface chip or device. The components of the computer can be connected in any suitable manner, such as via a wired media (e.g., a physical system bus 580, Ethernet connection, or any other wire transfer technology) or wirelessly (e.g., Bluetooth®, Wi-Fi®, or any other wireless technology).

Software module 550, which can be stored as executable instructions in storage 540 and executed by processor(s) 510, can include, for example, an operating system and/or the processes that embody the functionality of the methods of the present disclosure (e.g., as embodied in the devices as described herein).

Software module 550 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 540, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

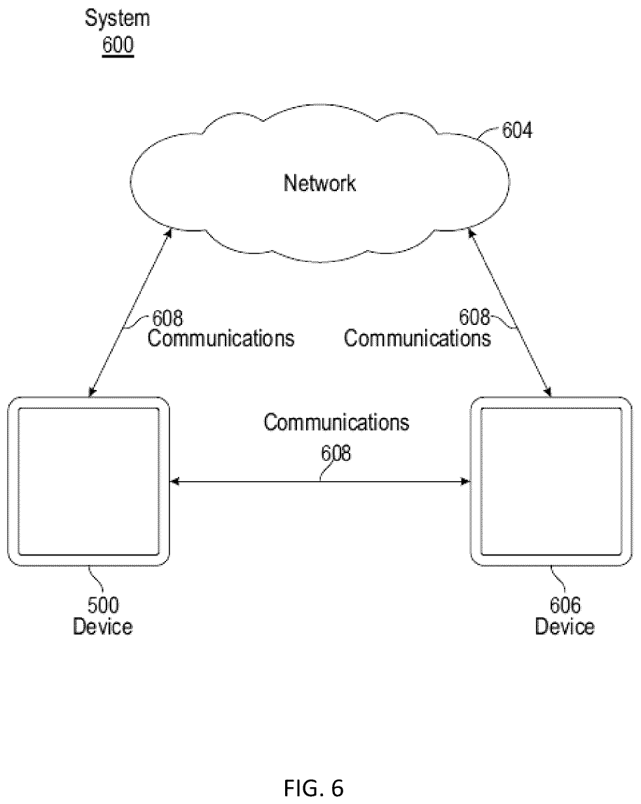

Device 500 may be connected to a network (e.g., network 604, as shown in FIG. 6 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can be implemented using any operating system, e.g., an operating system suitable for operating on the network. Software module 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 510.

Device 500 can further include a sequencer 570, which can be any suitable nucleic acid sequencing instrument.

FIG. 6 illustrates an example of a computing system in accordance with one embodiment. In system 600, device 500 (e.g., as described above and illustrated in FIG. 5) is connected to network 604, which is also connected to device 606. In some embodiments, device 606 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 500 and 606 may communicate, e.g., using suitable communication interfaces via network 604, such as a Local Area Network (LAN), Virtual Private Network (VPN), or the Internet. In some embodiments, network 604 can be, for example, the Internet, an intranet, a virtual private network, a cloud network, a wired network, or a wireless network. Devices 500 and 606 may communicate, in part or in whole, via wireless or hardwired communications, such as Ethernet, IEEE 802.11b wireless, or the like. Additionally, devices 500 and 606 may communicate, e.g., using suitable communication interfaces, via a second network, such as a mobile/cellular network. Communication between devices 500 and 606 may further include or communicate with various servers such as a mail server, mobile server, media server, telephone server, and the like. In some embodiments, Devices 500 and 606 can communicate directly (instead of, or in addition to, communicating via network 604), e.g., via wireless or hardwired communications, such as Ethernet, IEEE 802.11b wireless, or the like. In some embodiments, devices 500 and 606 communicate via communications 608, which can be a direct connection or can occur via a network (e.g., network 604).

One or all of devices 500 and 606 generally include logic (e.g., http web server logic) or are programmed to format data, accessed from local or remote databases or other sources of data and content, for providing and/or receiving information via network 604 according to various examples described herein.

### EXAMPLES

### Prophetic Example 1—ML-Based Prediction of EGFR Mutations in Images of Lung Adenocarcinomas

An exemplary ML training workflow could be based on, for example, data generated using an existing National Comprehensive Cancer Network (NCCN) guideline-based driver mutation detection pipeline. The methods of the present disclosure are directed to determining the presence of driver alterations in tissue specimens from pathology whole slide images. A cohort of slide images of lung tissue specimens collected from lung adenocarcinoma patients may be selected from the driver mutation detection pipeline database.

As a first step, a data set of lung adenocarcinoma specimens, each with a corresponding NGS result indicating the presence of a NCCN-guideline driver mutation or the absence of all driver mutations in the sample.

As a second step, a random sample of lung adenocarcinoma slide images may be selected and tissue morphology labeled image patches may be extracted from these slide images using a trained tissue morphology classifier (trained, for example, to identify and label image patches corresponding to tumor, normal, necrotic, stromal, and/or immune tissue regions).

As a third step, binary tissue masks are generated to define tissue regions across the whole slide images of the lung specimens and isolate tumor-only image patch regions.

As a fourth step, a random sub-sampling of the tumor-only image patches, plus a set of buffer region image patches, may be identified where the buffer region image patches comprise, for example, all image patches within or touching an n pixel region along the outer edge of the tumor region.

Finally, a machine learning model (e.g., a convolutional neural network) may be trained to learn predict a gene alteration status of interest (e.g., the presence or absence of an EGFR driver mutation) using the set of tumor-only and buffer region image patches.

### EXEMPLARY IMPLEMENTATIONS

Exemplary implementations of the methods and systems described herein include:

1. A method for predicting a gene alteration state in a sample, the method comprising:
receiving, at one or more processors, a plurality of image patches derived from at least one slide image of the sample, wherein each image patch of the plurality is associated with a tissue phenotype classification label;
identifying, using the one or more processors, a set of tumor region image patches from the plurality of image patches based on the tissue phenotype classification label for each image patch of the plurality of image patches;
identifying, using the one or more processors, a set of buffer region image patches from the plurality of image patches based on the identified set of tumor region patches and a set of proximity criteria used to define buffer region image patches;
inputting, using the one or more processors, the set of tumor region image patches and the set of buffer region image patches into a gene alteration state classification model to obtain a prediction of a presence of at least one gene alteration state for the tissue sample; and outputting, using the one or more processors, the prediction of a presence of the at least one gene alteration state for the sample.

2. The method of clause 1, further comprising generating the plurality of image patches by inputting, using the one or more processors, the plurality of image patches into a tissue phenotype classification model, wherein the tissue phenotype classification model is configured to classify each image patch and output a tissue phenotype classification label for the image patch.

3. The method of clause 1 or clause 2, wherein identifying the set of tumor region image patches comprises applying a binary mask based on tissue phenotype classification label to the plurality of image patches.

4. The method of any one of clauses 1 to 3, wherein the set of proximity criteria comprises an adjacency criterion, a distance criterion, or any combination thereof.

5. The method of any one of clauses 1 to 4, wherein identifying the set of buffer region image patches based on the identified set of tumor region patches and the set of proximity criteria comprises identifying a set of n nearest neighboring, non-tumor region image patches adjacent to a tumor region indicated by the identified set of tumor region patches.

6. The method of clause 5, wherein n is greater than or equal to 8, 10, 12, 14, 16, 18, 20, 40, 60, 80, 100, 120, 140, 160, 180, or 200 non-tumor region image patches.

7. The method of any one of clauses 1 to 4, wherein identifying the set of buffer region image patches based on the identified set of tumor region patches and the set of proximity criteria comprises identifying a set of n neighboring, non-tumor region image patches that are within a specified number of image pixels, m, of an edge of a tumor region indicated by the identified set of tumor region patches.

8. The method of clause 7, wherein m is greater than or equal to 1, 2, 3, 4, 5, 6, 7, 8, 9, 10, 20, 30, 40, 50, 60, 70, 80, 90, or 100 image pixels.

9. The method of any one of clauses 1 to 4, wherein identifying the set of buffer region image patches based on the identified set of tumor region patches and the set of proximity criteria comprises identifying a set of n neighboring, non-tumor region image patches that are within a predefined distance, d, of an edge of a tumor region indicated by the identified set of tumor region patches.

10. The method of clause 9, wherein d is greater than or equal to 1, 2, 3, 4, 5, 6, 7, 8, 9, 10, 20, 30, 40, 50, 60, 70, 80, 90, or 100 micrometers.

11. The method of any one of clauses 2 to 10, wherein the tissue phenotype classification model comprises a supervised machine learning model.

12. The method of clause 11, wherein the supervised machine learning model comprises a neural network.

13. The method of clause 12, wherein the neural network model comprises a convolutional neural network.

14. The method of any one of clauses 2 to 13, wherein the tissue phenotype classification model is trained on a plurality of annotated image patches (training image patches) derived from a plurality of slide images of samples from a cohort of subjects diagnosed with a disease of interest.

15. The method of clause 14, wherein the disease of interest is a cancer.

16. The method of any one of clauses 1 to 15, wherein the tissue phenotype classification label corresponds to one of a plurality of tissue phenotypes comprising a tumor tissue phenotype, a normal tissue phenotype, a necrotic tissue phenotype, a stromal tissue phenotype, an immune tissue phenotype, or any combination thereof.

17. The method of any one of clauses 1 to 16, wherein the gene alteration state classification model comprises a supervised machine learning model.

18. The method of clause 17, wherein the supervised machine learning model comprises a neural network.

19. The method of clause 18, wherein the neural network model comprises a convolutional neural network.

20. The method of any one of clauses 1 to 19, wherein the gene alteration state classification model is trained on tumor region and buffer region image patches derived from slide images and associated gene alteration state data for a plurality of samples from a cohort of subjects diagnosed with a disease of interest.

21. The method of clause 20, wherein the disease of interest is a cancer.

22. The method of clause 20 or clause 21, wherein the associated gene alteration state data comprises nucleic acid sequence data that indicates a presence of at least one gene alteration state in the samples from the cohort of subjects.

23. The method of clause 22, wherein the nucleic acid sequence data is derived from a next generation sequencing method.

24. The method of clause 23, wherein the next generation sequencing method comprises a targeted sequencing method.

25. The method of any one of clauses 1 to 24, wherein the prediction of at least one gene alteration state for the sample comprises a prediction of a presence of at least 1, 2, 3, 4, 5, 6, 7, 8, 9, or 10 gene alterations in the sample.

26. The method of any one of clauses 1 to 25, wherein the prediction of at least one gene alteration state comprises a prediction of a presence of at least 1, 2, 3, 4, 5, 6, 7, 8, 9, or 10 driver mutations in the sample.

27. The method of clause 25 or clause 26, wherein the prediction of at least one gene alteration state for the sample comprises a prediction of a presence of an EGFR alteration, ALK alteration, ROS1 alteration, NTRK1 alteration, NTRK2 alteration, NTRK3 alteration, KRAS alteration, G12C alteration, or any combination thereof in the sample.

28. The method of any one of clauses 2 to 27, further comprising generating the image patches of the plurality by processing the at least one slide image of the sample using an image segmentation algorithm.

29. The method of any one of clauses 1 to 28, wherein each image patch of the plurality of image patches is of a uniform, predetermined shape and size.

30. The method of clause 29, wherein the uniform, predetermined shape is square or rectangular.

31. The method of clause 29 or clause 30, wherein the uniform, predetermined size ranges from 32 pixels to 2048 pixels in at least one dimension.

32. The method of any one of clauses 1 to 31, wherein the at least one slide image of the sample comprises 1, 2, 3, 4, 5, 6, 7, 8, 9, or 10 slide images of the sample.

33. The method of any one of clauses 1 to 32, wherein the at least one slide image of the sample comprises a pathology slide image of the sample.

34. The method of any one of clauses 1 to 33, wherein the sample comprises a tissue sample.

35. The method of clause 33, wherein the tissue sample comprises a tissue biopsy sample.

36. The method of clause 33, wherein the tissue sample comprises a needle core biopsy sample.

37. A system comprising:
one or more processors; and
a memory communicatively coupled to the one or more processors and configured to store instructions that, when executed by the one or more processors, cause the system to perform the method of any one of clauses 1 to 36.

38. A non-transitory computer-readable storage medium storing one or more programs, the one or more programs comprising instructions, which when executed by one or more processors of a system, cause the system to perform the method of any one of clauses 1 to 36.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method for predicting a gene alteration state in a sample, the method comprising:
receiving, at one or more processors, a plurality of image patches derived from at least one slide image of the sample, wherein each image patch of the plurality is associated with a tissue phenotype classification label;
identifying, using the one or more processors, a set of tumor region image patches from the plurality of image patches based on the tissue phenotype classification label for each image patch of the plurality of image patches;
identifying, using the one or more processors, a set of buffer region image patches from the plurality of image patches based on the identified set of tumor region patches and a set of proximity criteria used to define buffer region image patches;
inputting, using the one or more processors, the set of tumor region image patches and the set of buffer region image patches into a gene alteration state classification model to obtain a prediction of a presence of at least one gene alteration state for the tissue sample; and
outputting, using the one or more processors, the prediction of a presence of the at least one gene alteration state for the sample.

2. The method of claim 1, further comprising generating the plurality of image patches by inputting, using the one or more processors, the plurality of image patches into a tissue phenotype classification model, wherein the tissue phenotype classification model is configured to classify each image patch and output a tissue phenotype classification label for the image patch.

3. The method of claim 1, wherein identifying the set of tumor region image patches comprises applying a binary mask based on tissue phenotype classification label to the plurality of image patches.

4. The method of claim 1, wherein the set of proximity criteria comprises an adjacency criterion, a distance criterion, or any combination thereof.

5. The method of claim 1, wherein identifying the set of buffer region image patches based on the identified set of tumor region patches and the set of proximity criteria comprises identifying a set of n nearest neighboring, non-tumor region image patches adjacent to a tumor region indicated by the identified set of tumor region patches.

6. The method of claim 1, wherein identifying the set of buffer region image patches based on the identified set of tumor region patches and the set of proximity criteria comprises identifying a set of n neighboring, non-tumor region image patches that are within a specified number of image pixels, m, of an edge of a tumor region indicated by the identified set of tumor region patches.

7. The method of claim 1, wherein identifying the set of buffer region image patches based on the identified set of tumor region patches and the set of proximity criteria comprises identifying a set of n neighboring, non-tumor region image patches that are within a predefined distance, d, of an edge of a tumor region indicated by the identified set of tumor region patches.

8. The method of claim 2, wherein the tissue phenotype classification model comprises a supervised machine learning model.

9. The method of claim 2, wherein the tissue phenotype classification model is trained on a plurality of annotated image patches (training image patches) derived from a plurality of slide images of samples from a cohort of subjects diagnosed with a disease of interest.

10. The method of claim 1, wherein the tissue phenotype classification label corresponds to one of a plurality of tissue phenotypes comprising a tumor tissue phenotype, a normal tissue phenotype, a necrotic tissue phenotype, a stromal tissue phenotype, an immune tissue phenotype, or any combination thereof.

11. The method of claim 1, wherein the gene alteration state classification model comprises a supervised machine learning model.

12. The method of claim 1, wherein the gene alteration state classification model is trained on tumor region and buffer region image patches derived from slide images and associated gene alteration state data for a plurality of samples from a cohort of subjects diagnosed with a disease of interest.

13. The method of claim 12, wherein the associated gene alteration state data comprises nucleic acid sequence data that indicates a presence of at least one gene alteration state in the samples from the cohort of subjects.

14. The method of claim 1, wherein the prediction of at least one gene alteration state for the sample comprises a prediction of a presence of at least 1, 2, 3, 4, 5, 6, 7, 8, 9, or 10 gene alterations in the sample.

15. The method of claim 1, wherein the prediction of at least one gene alteration state comprises a prediction of a presence of at least 1, 2, 3, 4, 5, 6, 7, 8, 9, or 10 driver mutations in the sample.

16. The method of claim 14, wherein the prediction of at least one gene alteration state for the sample comprises a prediction of a presence of an EGFR alteration, ALK alteration, ROS1 alteration, NTRK1 alteration, NTRK2 alteration, NTRK3 alteration, KRAS alteration, G12C alteration, or any combination thereof in the sample.

17. The method of claim 2, further comprising generating the image patches of the plurality by processing the at least one slide image of the sample using an image segmentation algorithm.

18. The method of claim 1, wherein each image patch of the plurality of image patches is of a uniform, predetermined shape and size.

19. The method of claim 1, wherein the at least one slide image of the sample comprises a pathology slide image of the sample.

20. The method of claim 1, wherein the sample comprises a tissue sample.

21. A system comprising:
one or more processors; and
a memory communicatively coupled to the one or more processors and configured to store instructions that, when executed by the one or more processors, cause the system to perform the method of claim 1.

22. A non-transitory computer-readable storage medium storing one or more programs, the one or more programs comprising instructions, which when executed by one or more processors of a system, cause the system to perform the method of claim 1.
